(19) United States
(12) Patent Application Publication
(10) Pub. No.: US 2009/0312243 A1
(43) Pub. Date: Dec. 17, 2009

(54) TREATMENT OF INFLAMMATORY BOWEL DISEASE (IBD) WITH ANTI-ANGIOGENIC COMPOUNDS

(76) Inventors: Silvio Danese, Milan (IT); Andrew P. Mazar, San Diego, CA (US); Claudio Fiocchi, Shaker Heights, OH (US)

Correspondence Address: BROWDY AND NEIMARK, P.L.L.C.;624 NINTH STREET, NW, SUITE 300, WASHINGTON, DC, 20001-5303, US

(21) Appl. No.: 11/914,380

(22) PCT Filed: May 12, 2006
(86) PCT No.: PCT/US06/18463

§ 371 Date: Sep. 18, 2008

Related U.S. Application Data
(60) Provisional application No. 60/679,977, filed on May 12, 2005.

(51) Int. Cl. A61K 38/03; A61K 38/16; A61P 1/00

(52) U.S. Cl. 514/12; 514/13; 514/14; 514/15; 514/16; 514/17

(57) ABSTRACT

Inhibitors of angiogenesis are disclosed as being useful therapeutics for treating various aspects of inflammatory bowel disease, in particular Crohn's Disease. A method for decreasing the magnitude of intestinal inflammation or inflammatory infiltrate in bowel tissue, a method for lowering systemic or gut-associated levels of a proinflammatory cytokine in a subject, a method for reducing microvessel density in fixed bowel tissue sections and a method for treating an inflammatory bowel disease are disclosed. Preferred agents to achieve the foregoing are pentapeptides that include Pro-His-Ser-Cys-Asn (SEQ ID NO:1) and variants or derivatives thereof.

### BACKGROUND OF THE INVENTION

1. Field of the Invention

The invention in the field of biochemistry and medicine relates to the discovery that inhibitors of angiogenesis are useful therapeutics for inflammatory bowel disease (IBD) in particular Crohn's Disease (CrD), and provides novel methods for treating these conditions.

2. Description of the Background Art

Chronic Inflammatory Disease and Animal Models

Inflammatory conditions, particularly chronic inflammatory diseases, are of particular importance in clinical medicine. These diseases, caused by actions of the immune system, involve inappropriate activation of T cells, expression of regulatory cytokines and chemokines, loss of immune tolerance, and the like. Examples of autoimmune and/or chronic inflammatory diseases are multiple sclerosis, inflammatory bowel diseases (IBD), joint diseases such as rheumatoid arthritis, systemic lupus erythematosus. Some of these diseases are rather organ/tissue-specific as follows: intestine (CrD), skin (psoriasis), pancreatic islet or β cells (insulin dependent diabetes mellitus (IDDM)), salivary glands (Sjogren's disease), skeletal muscle (myasthenia gravis), the thyroid (Hashimoto's thyroiditis; Graves' Disease), the anterior chamber of the eye (uveitis), and various cardiovascular diseases.

Inflammatory bowel disease (IBD) is a collective term used to describe two intestinal disorders whose etiology is not completely understood: Crohn's disease (CrD) and ulcerative colitis. The course and prognosis of IBD, which occurs worldwide and afflicts several million people, varies widely. Onset of IBD is predominant in young adulthood and presents typically with diarrhea, abdominal pain, and fever. Anemia and weight loss are also common signs of IBD. Between 10% and 15% of people with IBD require surgery over a ten year period. Patients with IBD are also at increased risk for the development of intestinal cancer. These diseases are accompanied by a high frequency of psychological symptoms, including anxiety and depression.

Unfortunately, new therapies for IBD are few, and both diagnosis and treatment have been hampered by a lack of detailed knowledge of the etiology. A combination of genetic factors, exogenous triggers and endogenous microflora can contribute to the immune-mediated damage of intestinal mucosa. Bacteria have been implicated in initiation and progression of CrD based on the finding that intestinal inflammation frequently responds to antibiotics. Common intestinal colonists and novel pathogens have been implicated in CrD, either because of direct detection or disease-associated anti-microbial immune responses. In many genetically susceptible animal models of chronic colitis, luminal microorganisms appear to be a necessary cofactor for disease as animals housed under germ-free conditions do not develop the disease.

The initiating step in autoimmune disease pathology is often obscure in humans where the diseases are largely sporadic, and symptoms may appear years after the first pathogenic T cell is activated. It has therefore been difficult to design effective therapies to block induction of disease. In contrast, there are common features in many of the later stages of these diseases. Inflammation at the disease site/target organ is typically present, caused by the release of inflammatory (also termed “proinflammatory”) cytokines by T cells and by other cells that contribute to the activation steps and effector pathways of immune/inflammatory processes. These cells include macrophages, dendritic cells and their precursors, B lymphocytes and plasma cells and NK cells (including NKT cells). These reactions often involved destruction of “target” cells and tissue damage.

Studies using murine models of experimental chronic inflammation are helping to define nature of the immunological dysregulation that initiates inflammation and leads to destruction of specific end organs. See, for example, Mombaerts et al. Cell, 1993, 75:274-82; Tarrant et al., J Immunol, 1998. 161:122-127; Powrie et al, Immunity, 1994, 1:553-562; Hong et al., J Immunol, 1999. 162:7480-91; Horak, Clin Immunol Immunopathol, 1995, 76(3 Pt 2):S172-173; Ehrhardt et al. J Immunol, 1997. 158:566-73; Davidson et al., J Immunol, 1998, 161:3143-9; Kuhn et al. Cell, 1993. 75(2):263-74; Neurath et al., J Exp Med, 1995. 182:1281-90). A recent review of mucosal models of inflammation, which is incorporated by reference in its entirety, is Strober, W et al., 2002, Annu. Rev. Immunol. 20:495-54. Animal models have provided very useful tools for studying the panoply of interactions, as noted above. One hallmark of the better of these models is that the histopathology and pathophysiology resembles that of the parallel human conditions, further enhancing the models' utility in testing novel treatment strategies. In the case of IBD this development has not been uniform, and most emphasis has been placed on modulation of immune mechanisms (Blumberg R S et al., 1999, Current Opin Immunol. 11:648-656; Strober et al., supra) and recently of the enteric flora (Sartor R B, 2001, Curr Opin Gastroenterol. 4:324-330).

Interleukin 10 (IL-10) and Chronic Inflammatory Disease

It has been known for some years that interleukin-10 (IL-10) affects the growth and differentiation of many hemopoietic cell types in vitro and is a particularly potent suppressor of macrophage and T cell functions. One way this was shown was by creating IL-10-deficient (knockout or KO) mutant mice by gene targeting (Kuhn R et al., 1993, Cell 75:263-74). In these mice, lymphocyte development and antibody responses are normal, but most animals are growth retarded and anemic and suffer from chronic enterocolitis. Alterations in the intestine include extensive mucosal hyperplasia, inflammatory reactions, and aberrant epithelial expression of major histocompatibility complex (MHC) class II molecules. In contrast, if these IL-10 KO mutants are kept under specific pathogen-free conditions, they develop only localized inflammation (limited to the proximal colon). It was therefore concluded that (1) bowel inflammation in these mutants originated from uncontrolled immune responses stimulated by enteric antigens and (2) IL-10 is an essential (negative) regulator in the intestinal tract.

More recently, Takeda, K et al., 1999, Immunity 10:39-49, reported on mice with a cell type-specific disruption of the Stat3 gene in macrophages and neutrophils. These Stat-3 KO mice were highly susceptible to endotoxin (=lipopolysaccharide or “LPS”)-mediated shock and produced increased levels of inflammatory cytokines such as TNFα, IL-1, IFNγ, and IL-6. The authors concluded that the LPS-induced production of these cytokines was augmented due to the loss of the suppressive effects of IL-10 on inflammatory cytokine production. These mice showed an immune response that was “polarized” toward the Th1-type and developed chronic enterocolitis with age. It is evident that Stat3 plays a critical role in the “deactivation” of macrophages and neutrophils mainly mediated by IL-10. The IL-10 KO model served the model of choice in exemplifying the present invention.

Bhan A K et al., 1999, Immunol Rev 169:195-207 reviewed studies of colitis in transgenic (Tg) and KO animal models that have been used to study the development of mucosal inflammation in IBD. Genetic and environmental factors, particularly the normal enteric flora, were factors in the development of mucosal inflammation, as stated above. Normal mucosal homeostasis was disrupted by cytokine imbalance, abrogation of oral tolerance, breach of epithelial barriers, and loss of immunoregulatory cells. Some but not all immunodeficiencies, in the appropriate setting, led to colitis. CD4+ T cells have been identified as the pathogenic lymphocytes in colitis, and can mediate inflammation by either the Th1 or the Th2 pathway. The Th1 pathway dominates most colitis models (and human CrD). In contrast, the colitis observed in mice that were KO's of the T cell receptor α chain (TCRα KO mice) shared many features of ulcerative colitis including the dominance of Th2 pathway in colonic inflammation. Such models are important for the development of therapeutic strategies to treat IBD. In a later review, the same group (Mizoguchi A et al., 2003, Inflamm Bowel Dis. 9:246-259) noted that exaggerated immune responses to normal enteric microflora are involved in the initiation and perpetuation of chronic intestinal inflammation. A major pathway involves development of “acquired” immune responses by the interactions of CD4+ TCRαβ T cells with antigen-presenting cells (dendritic cells). Immunoregulatory cells, including Tr1 cells, Th3 cells, and CD4+ CD25+T cells as well as B cells, directly or indirectly affected the activated T cell responses.

IL-10-deficient (IL-10KO) mice infected with Toxoplasma gondii succumbed to a T-cell-mediated shock-like reaction characterized by the overproduction of IL-12 and IFNγ associated with widespread liver necrosis (Villegas E N et al., 2000, Infect Immun. 68:2837-44). Infection of mice with T. gondii resulted in increased expression of B7 and CD40 costimulatory molecules that was similar in wild-type and IL-10KO mice. In vivo blockade of two sets of costimulatory interactions (CD28-B7 or CD40-CD40L) following T. gondii infection of these mice did not affect serum levels of IFNγ or IL-12, nor did it prevent death in these mice. However, when both pathways were blocked, the IL-10 KO mice survived the acute phase of infection and had reduced serum IFNγ and alanine transaminase as well as decreased expression of inducible nitric oxide synthase in liver and spleen. Whereas blockade of the CD40-CD40L interaction had minimal effects on cytokine production in parasite-specific recall responses, blockade of the CD28-B7 interaction resulted in decreased production of IFNγ (but not IL-12). Further reduction of IFNγ production occurred when both costimulatory pathways were blocked. The authors concluded that, in the absence of IL10-mediated regulation, both CD28-B7 and CD40-CD40L interactions are involved in the development of infection-induced immunopathology.

The progression from the acute to the chronic phase of IBD has not been well characterized in animal models and cannot be easily evaluated in patients. Spencer D M et al Gastroenterology 122:94-105 (2002) reported a longitudinal study of changes in the mucosal immune response in an experimental model of colitis. Severity of colitis, body mass, stool consistency and blood content, serum amyloid A, and tissue histology were examined in IL-10-deficient mice over 35 weeks. The corresponding production of IL-12, IL-18, IFNγ, TNFα, IL-4, and IL-13 by lamina propria mononuclear cells in the inflamed intestine was measured. Administration of a neutralizing anti-IL-12 monoclonal antibody (mAb) antibody at distinct times during disease progression permitted evaluation of the therapeutic potential of this compound. The clinical manifestations coupled with the form of intestinal inflammation delineated an early phase of colitis (10-24 weeks), characterized by a progressive increase in disease severity, followed by a late phase (>25 weeks), in which chronic inflammation persisted indefinitely. Lamina propria mononuclear cells from mice with early disease synthesized progressively more IL-12 and IFNγ, whereas production of both cytokines declined dramatically and returned to pre-disease levels in the late phase. Consistent with this pattern, the neutralizing anti-IL-12 reversed early, but not late, disease. In contrast, IL-4 and IL-13 production increased progressively from pre- to early to late disease. It was concluded that colitis that develops in IL-10-deficient mice evolves into two distinct phases. IL-12 plays a pivotal role in early colitis, whereas other immune mechanisms, presumably mediated by IL-4 and IL-13, predominate in late disease to sustain chronic inflammation.

The IL-10 KO mouse model of colitis has recently been validated further by Scheinin, T. et al., Clin Exp Immunol 133:38-43 (2003). These authors emphasized that a truly valuable model must respond to existing therapy in a way that resembles the response of human disease. Since refractory CrD was shown to respond well to anti-TNFα antibody therapy, the investigators examined the response of IL-10 KO mice to anti-TNFα therapy, and developed a new scoring system for the IL-10 KO mice, similar to the CrD “Activity Index” in humans. Stool samples were tested for cytokines and the findings compared with histology. They reported that anti-TNF antibody therapy starting at 4 weeks markedly ameliorated the disease, as judged by the clinical score or by gut histology. A marked diminution of inflammatory cytokines in stool samples was noted, adding a further accurate measure of clinical improvement. The authors concluded that this model is useful for evaluating other therapeutic modalities of relevance to CrD.

Angiogenesis (neoangiogenesis or neovascularization) is defined as the process of new capillary formation from pre-existing vasculature in adult tissues (Folkman J et al., 1992, J Biol Chem 267:10931-34). Angiogenesis is a fundamental constituent of biological processes, including growth, development and repair, but in the last three decades has emerged as a phenomenon essential for the growth of tumors, and its inhibition has been hailed as a cornerstone of cancer therapy (Folkman J, 1971, N Engl J Med 285:1182-1186). A milestone in this field has been the report that a monoclonal antibody against VEGF prolongs the survival of colorectal cancer patients (Hurwitz H et al., 2004, N Engl J Med 350:2335-2342). It is now appreciated that the importance of angiogenesis extends beyond cancer biology, as it has essential functions in non-neoplastic diseases as diverse as atherosclerosis, rheumatoid arthritis, diabetic retinopathy, psoriasis, airway inflammation, peptic ulcers, and Alzheimer disease (Gould V E et al., 2002, Human Pathol 33:1061-1063; McDonald D M., 2002, Am J Respir Crit Care Med. 164:S39-S45; Vagnucci A H et al., 2003, Lancet 361:605-608).

Pathological angiogenesis is almost invariably associated with some degree of inflammation and recently, IBD has been listed among the several inflammatory conditions in which vascular/microcirculatory phenomena and abnormal or excessive angiogenesis could play a role (Carmeliet P, 2003, Nature Med. 9:653-60; Laroux F S et al., 2001, Microcirculation 8:283-301; Hatoum O A et al, 2003, Am J Physiol Heart Circ Physiol 285:H1791-96). Only two recent reports touch upon angiogenesis in the context of animal models of IBD—both involving dextran sulfate sodium (DSS)-induced murine colitis. In the first study using in vivo confocal microscopy, the investigators observed diffuse hypervascularity and vessel tortuosity and dilation of mucosal capillaries several days after DSS administration (McLaren W J et al., 2002, Dig Dis Sci 47:2424-2433). The second study, evaluating the effect of cigarette smoke on inflammation-associated colon tumorigenesis, reported increased VEGF and angiogenesis in smoke-exposed animals in which colitis had been induced by DSS (Liu ESL et al., 2003, Carcinogenesis 24:1407-1413). However, there have been no reports of the impact of inhibiting angiogenesis on the pathophysiology of IBD or CrD. That is the subject of the present invention. Despite the progress noted above, there remains a need in the art for new and improved methods for treating this debilitating group of diseases, and the present inventors have made a significant step forward with the invention disclosed herein.

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention provides a method of inhibiting pathological events associated with IBD, particularly CrD, by providing to a subject in need of such inhibition, an effective amount of an anti-angiogenic compound.

Specifically, the invention is directed to a method for decreasing the magnitude of intestinal inflammation in bowel tissue of a subject with an IBD, comprising, administering to a subject in need of such treatment an effective amount of a pharmaceutical composition that comprises:

(a) a compound that inhibits angiogenesis; and
(b) a pharmaceutically acceptable carrier or excipient;
thereby decreasing the inflammation or infiltrate.

The invention also includes a method of lowering systemic or gut-associated levels of proinflammatory cytokines in a subject, comprising providing to a subject in need of such lowering, an effective amount of an anti-angiogenic pharmaceutical composition that comprises

(a) a compound that inhibits angiogenesis; and
(b) a pharmaceutically acceptable carrier or excipient;
thereby lowering the level of the proinflammatory cytokines.

The invention is also directed to method for reducing microvessel density, as determined in fixed bowel tissue sections, from a biopsy obtained from a subject with an IBD, comprising
(a) administering to a subject in need of such treatment an effective amount of a pharmaceutical composition that comprises
   (i) a compound that inhibits angiogenesis; and
   (ii) a pharmaceutically acceptable carrier or excipient;
(b) obtaining a bowel biopsy from the subject, and
(c) determining the microvessel density in the biopsy,
   wherein the administering of the compound results in a lower microvessel density compared to the microvessel density before receipt of the compound by the subject.

The invention includes a method for treating an IBD in a subject, comprising administering to a subject in need of such treatment an effective amount of a pharmaceutical composition that comprises

(a) an compound that inhibits angiogenesis; and
(b) a pharmaceutically acceptable carrier,
thereby treating the disease.

In the above method, the compound is a peptide of 5 to about 30 amino acid residues which comprises the amino acid sequence

| | |
|---|---|
| Xaa₁-Xaa₂-Xaa₃-Xaa₄-Xaa₅, | (SEQ ID NO: 81) |

wherein
Xaa₁ is Pro, Gly, Val, His, Iso, Phe, Tyr, or Trp;
Xaa₂ is His, Pro, Tyr, Asn, Glu, Arg, Lys, Phe, or Trp;
Xaa₃ is Ser, Thr, Ala, Tyr, Leu, His, Asn, or Glu;
Xaa₄ is L- or D-Cys, L- or D-homocysteine (Hcy), L- or D-penicillamine, any other amino acid having a —SH group or L- or D-His; and
Xaa₅ is Asn, Glu, Ser, Thr, His, or Tyr,
   or is a N- and C-terminally capped derivative of the peptide.

In another embodiment of the above method, the peptide has the amino acid sequence

| | |
|---|---|
| Xaa₁-His-Ser-Xaa₂-Asn, | (SEQ ID NO: 86) |

wherein Xaa₁ is Pro, His, or is not an amino acid, and Xaa₂ is L- or D-Cys, L- or D-Hcy, L- or D-penicillamine, any other amino acid having an —SH group, or D- or L-His.

In a preferred embodiment, the above peptide has the amino acid sequence

| | |
|---|---|
| Pro-His-Ser-Xaa-Asn, | (SEQ ID NO: 87) |

wherein X is L- or D-Cys, L- or D-Hcy, penicillamine any other amino acid having an —SH group, or D- or L-His.

A preferred peptide includes the amino acid sequence Pro-His-Ser-Cys-Asn (SEQ ID NO:1).

A most preferred antiangiogenic compound is a pentapeptide with the amino acid sequence Pro-His-Ser-Cys-Asn (SEQ ID NO:1).

In the above method, the peptide is preferably N-terminally capped, preferably with an acyl group, more preferably with an acetyl group. The peptide is preferably C-terminally capped with an amido group, more preferably with an amino group.

In a preferred embodiment of the above method, the subject is a human and the IBD is CrD.

The invention also includes first and second medical uses of an anti-angiogenic compound as described herein, for achieving any of the following aims or for the preparation of a medicament for achieving any of the following aims:
(a) decreasing the magnitude of intestinal inflammation or inflammatory infiltrate in bowel tissue of a subject with an IBD; and/or
(b) lowering systemic or gut-associated levels of proinflammatory cytokines in a subject,
(c) reducing microvessel density, as determined in fixed bowel tissue sections, in a biopsy obtained from a subject with an IBD; and/or
(d) treating an IBD in a subject.

In the above use, the compound may be is a peptide of 5 to about 30 amino acid residues which comprises the amino acid sequence Xaa₁-Xaa₂-Xaa₃-Xaa₄-Xaa₅ (SEQ ID NO:81),

wherein
Xaa₁ is Pro, Gly, Val, His, Iso, Phe, Tyr, or Trp;
Xaa₂ is His, Pro, Tyr, Asn, Glu, Arg, Lys, Phe, or Trp;
Xaa₃ is Ser, Thr, Ala, Tyr, Leu, His, Asn, or Glu;
L- or D-Cys, L- or D-Hcy, L- or D-penicillamine, any other amino acid having a —SH group or L- or D-His, and
Xaa₅ is Asn, Glu, Ser, Thr, His, or Tyr.
   or wherein the peptide is N- and C-terminally capped.

In another embodiment of the above use, the peptide has the sequence Xaa₁-His-Ser-Xaa₂-Asn (SEQ ID NO:86), wherein Xaa₁ is Pro, His, or is not an amino acid, and Xaa₂ is L- or D-Cys, L- or D-Hcy, L- or D-penicillamine, any other amino acid having a —SH group or L- or D-His. In a preferable use as above, the peptide has the amino acid sequence Pro-His-Ser-Xaa-Asn (SEQ ID NO:87), wherein X is L- or D-Cys, L- or D-Hcy, L- or D-penicillamine of L- or D-His. Most preferably, the peptide is an pentapeptide with the amino acid sequence Pro-His-Ser-Cys-Asn (SEQ ID NO:1). Also included is the use as above wherein the peptide is N-terminally capped with an acetyl group and is C-terminally capped with an amino group.

In a preferred use, the subject is a human. In a preferred use, the IBD is CrD

### BRIEF DESCRIPTION OF THE DRAWINGS

FIG. 1 is a graph showing that ATN-161 reduces established colitis measured by disease activity index (DAI). The DAI was calculated by scoring 1 point for the appearance of each of the following: ruffled fur, occult fecal blood as determined on a Hemoccult Sensa® card (Smith Kline Diagnostics, San Jose, Calif.), rectal prolapse <1 mm, and soft stool. The mice were scored an additional point for diarrhea or severe rectal prolapse >1 mm. The mean with standard error bars of the DAI are plotted for pretreatment and then at the end of each week of treatment (n=12 per group).

FIG. 2 is a graph summarizing the histologic grading for colitis at Week 6. Grading of intestinal inflammation was determined in a blinded fashion by three readers: no inflammation (0); modest numbers of infiltrating cells in the lamina propria (1); infiltration of mononuclear cells leading to separation of crypts and mild mucosal hyperplasia (2); massive infiltration with inflammatory cells accompanied by disrupted mucosal architecture, loss of goblet cells, and marked mucosal hyperplasia (3); all of the above plus crypt abscesses or ulceration (4).

FIGS. 3A and 3B are graphs showing that colon fragments from ATN-161 treated mice expressed lower levels of IL-12 and IL-6. Fragments of colon from mice treated with ATN-161 or ATN-163 were cultured in RPMI media +2% fetal bovine serum and 2.5% PSF (antibiotic-antimycotic). After 48 hours, supernatants were harvested and kept frozen at −80 C. FIG. 3A: IL-12(p40) was analyzed using an ELISA in which plates had been coated with the monoclonal antibody C15.6. FIG. 3B: IL-6 was analyzed using a commercially available ELISA kit specific for mouse IL-6 (R&D Systems, Minneapolis, Minn.).

FIG. 4 is a graph showing an analysis of microvessel density using anti-CD31 immunostaining. Immunostaining was performed using the mouse CD31 specific mAb MEC 13.3 (Becton-Dickinson, San Diego, Calif.). Morphometric analysis was carried out using an international consensus for the quantification of angiogenesis (Vermeulen P B et al., Eur J Cancer 32A:2474-84; Eur J Cancer 38:1564-15). In particular stained colonic sections were scanned at low power (40×) to detect the most vascularized area, after which at least 5 microphotographs at 200× magnification were taken in the mucosa and in the submucosa. The number of vessels/field (mean vascular density) was performed using Image Pro Plus Software (Media Cybernetics, Silver Spring, Md.).

### DESCRIPTION OF THE PREFERRED EMBODIMENTS

The present inventors have discovered that an anti-angiogenic compound, a derivatized pentapeptide having the sequence Pro-His-Ser-Cys-Asn (SEQ ID NO:1), referred to herein in single letter code as PHSCN, dramatically reduced symptoms of existing IBD in a murine model of CrD. They further conceived that inhibition of angiogenesis by compounds in addition to that exemplified here would yield similar results, making this “class” of compounds potentially powerful drugs for treating IBD, especially CrD.

Use of the pentapeptide PHSCN (SEQ ID NO:1) and its capped derivative Acetyl-PHSCN—NH₂ (abbreviated Ac-PHSCN—NH₂) are the preferred embodiments of the present invention. Ac-PHSCN—NH₂ is also referred to herein by its drug development abbreviation “ATN-161”. These molecules and other substitution variants thereof and a number of their pharmacological uses are described in the following patents of D. Livant and publications of Livant et al. which are incorporated by reference in their entirety: U.S. Pat. Nos. 5,840,514, 5,989,850, 6,001,965, 6,025,150, 6,140,068, 6,331,409, 6,472,369, 6,576,440 and 6,841,355; Livant et al., Cancer Res, 2000, 60309-20.

Specific integrins, a class of cell-surface molecules, have been identified as being crucial for mediating the angiogenic response of endothelial cells (ECs). The integrins αVβ3, αVβ5 and α5β1 have been shown to play important roles in neoplastic and non-neoplastic angiogenesis by promoting EC migration, proliferation and survival (Brooks P C et al., 1994, Cell 79:1157-64; Brooks P C et al., 1995, J Clin Invest 96:1815-22; Kerr J S et al., 1999, Anticancer Res 19:959-68; Kerr J S et al., 2000, Expert Opin Investig Drugs 9:1271-79). Therefore, endothelial-specific integrins have become a promising target for anti-angiogenic approaches in antineoplastic regimens and in the treatment of nonmalignant angiogenic disorders (Kumar C C et al., 2001, Cancer Res 61:2232-2338; Kumar C C et al., Adv Exp Med Biol 476:169-80; Klotz O et al., 2000, Graefes Arch Clin Exp Ophthalmol 238:88-93; Storgard C M et al., 1999, J Clin Invest 103:47-54).

The present invention focuses on a particular anti-angiogenic compound as a preferred embodiment. This compound, termed ATN-161 as noted above, is in fact a derivatized (capped) integrin antagonist pentapeptide PHSCN (SEQ ID NO:1) and was selected, among other reasons, because of the activity it manifested in studies conducted by one of the present inventors (A. Mazar) with other colleagues (Stoeltzing, O et al., 2003, Int. J. Cancer: 104:496-503). In contrast to most integrin antagonists, ATN-161 is unique in that it is not based on an RGD sequence and does not affect cell adhesion. The sequence of this peptide was derived from a 5 residue sequence of fibronectin (PHSRN, SEQ ID NO:2), known as the “synergy region” which potentiates the binding of fibronectin to α5β1. ATN-161 has a sequence that is an Arg→Cys substitution of SEQ ID NO:2. ATN-161 can act by interfering with this integrin interaction. PHSCN (SEQ ID NO:1) and other useful substitution variants have been described in the Livant patents (supra) and Livant et al., 2001, supra. White et al, 2001, J Immunol 167:5362-66, showed that the PHSCN peptide may inhibit expression of pro-angiogenic CXC chemokines by monocytes that had been plated on fibronectin. Other studies by Mazar and colleagues showed that ATN-161 interacts with the N-terminus of the β1-domain of integrin α5β1, which may lock this integrin in an inactive conformation. The inhibitory action of ATN-161 on various aspects of tumor growth or survival are believed to be mediated at least in part by its directed effect on endothelial cells as opposed to tumor cells, because ATN-161 significantly reduced the in vivo growth of xenografted human colon cancer cells (HT29) that do not express integrin α5β1 (Stoeltzing O et al., 2001, Clin Cancer Res 7:3656 S). See, also the following published abstracts: Plunkett, M L et al., 2002, “A novel anti-angiogenic/anti-metastatic peptide, ATN-161 (Ac-PHSCN—NH2), which targets multiple fully activated integrins including α5β1 and αvβ3, leads to increased anti-tumor activity and increased survival in multiple tumor models when combined with chemotherapy.” Europ J Canc 38 (Suppl. 7):79; Plunkett M L et al., 2002, “Dose and schedule optimization of a novel anti-angiogenic/anti-metastatic peptide, ATN-161 (Ac-PHSCN—NH2), which targets multiple fully activated integrins including α5β1 and αvβ3.” Europ J Canc 38 (Suppl. 7):82; Doñate, F et al., 2003, “ATN-161 (Ac-PHSCN—NH₂) has potent anti-angiogenic activity through multiple mechanisms of action and localizes to newly formed blood vessels in vivo.” Proc. Amer Asoc Canc Res #44:63.

In a more recent study of ATN-161, one of the present inventors (Mazar) and his colleagues found this pentapeptide derivative to (1) inhibited tumor angiogenesis and, (2) enhance the efficacy of the classic chemotherapeutic drug, 5-fluorouracil (Stoeltzing et al., 2003 supra).

### Peptides Based on the PHSRN (SEQ ID NO:2) Sequence of Fibronectin

In a preferred embodiment, the method employs a peptide comprising, consisting essentially of, or consisting of, the sequence PHSCN (SEQ ID NO:1) and substitution or addition variants of SEQ ID NO:1. The peptide may be longer than five amino acids, that is, an addition variant, but is preferably no longer than about 30 amino acids. The most preferred peptide compositions for use in this invention are pentapeptides.

All amino acids listed herein are L-amino acids unless it is specifically stated that they are D-amino acids. It should be understood that the present invention includes embodiments wherein one or more of the L-amino acids is replaced with its D isomer.

Non-limiting examples of antiangiogenic peptides that include the sequence PHSCN (SEQ ID NO:1) and that therapeutic or other beneficial activity against the IBD, for example, the IBD of IL-10 KO mice, are listed below. (These include addition variants of SEQ ID NO:1 at the C-terminus, N-terminus or both.)

| | |
|---|---|
| PHSCN | (SEQ ID NO: 2) |
| PHSCNS | (SEQ ID NO: 3) |
| PHSCNSI | (SEQ ID NO: 4) |
| PHSCNSIT | (SEQ ID NO: 5) |
| PHSCNSITL | (SEQ ID NO: 6) |
| PHSCNSITLT | (SEQ ID NO: 7) |
| PHSCNSITLTN | (SEQ ID NO: 8) |
| PHSCNSITLTNL | (SEQ ID NO: 9) |
| PHSCNSITLTNLT | (SEQ ID NO: 10) |
| PHSCNSITLTNLTP | (SEQ ID NO: 11) |
| PHSCNSITLTNLTPG | (SEQ ID NO: 12) |
| EHFSGRPREDRVPHSCN | (SEQ ID NO: 13) |
| PEHFSGRPREDRVPHSCN | (SEQ ID NO: 14) |
| HFSGRPREDRVPHSCN | (SEQ ID NO: 15) |
| FSGRPREDRVPHSCN | (SEQ ID NO: 16) |
| SGRPREDRVPHSCN | (SEQ ID NO: 17) |
| GRPREDRVPHSCN | (SEQ ID NO: 18) |
| RPREDRVPHSCN | (SEQ ID NO: 19) |
| PREDRVPHSCN | (SEQ ID NO: 20) |
| REDRVPHSCN | (SEQ ID NO: 21) |
| EDRVPHSCN | (SEQ ID NO: 22) |
| DRVPHSCN | (SEQ ID NO: 23) |
| RVPHSCN | (SEQ ID NO: 24) |
| VPHSCN | (SEQ ID NO: 25) |
| PPSCN | (SEQ ID NO: 26) |
| PEHFSGRPREDRVPHSCNSITLTNLTPG | (SEQ ID NO: 27) |

Examples of substitution variants of the foregoing pentapeptide sequences useful in this invention that may be used as pentapeptides, or included in longer peptides, are:

| | | | | | |
|---|---|---|---|---|---|
| -HHSCN- | (SEQ ID NO: 28) | -HPSCN- | (SEQ ID NO: 29) | -PHTCN- | (SEQ ID NO: 30) |
| -HHTCN- | (SEQ ID NO: 31) | -HPTCN- | (SEQ ID NO: 32) | -PHSNN- | (SEQ ID NO: 33) |
| -HHSNN- | (SEQ ID NO: 34) | -HPSNN- | (SEQ ID NO: 35) | -PHTNN- | (SEQ ID NO: 36) |
| -HHTNN- | (SEQ ID NO: 37) | -HPTNN- | (SEQ ID NO: 38) | -PHSKN- | (SEQ ID NO: 39) |
| -HHSKN- | (SEQ ID NO: 40) | -HPSKN- | (SEQ ID NO: 41) | -PHTKN- | (SEQ ID NO: 42) |
| -HHTKN- | (SEQ ID NO: 43) | -HPTKN- | (SEQ ID NO: 44) | -PHSCR- | (SEQ ID NO: 45) |
| -HHSCR- | (SEQ ID NO: 46) | -HPSCR- | (SEQ ID NO: 47) | -PHTCR- | (SEQ ID NO: 48) |
| -HHTCR- | (SEQ ID NO: 49) | -HPTCR- | (SEQ ID NO: 50) | -PHSNR- | (SEQ ID NO: 51) |
| -HHSNR- | (SEQ ID NO: 52) | -HPSNR- | (SEQ ID NO: 53) | -PHTNR- | (SEQ ID NO: 54) |
| -HHTNR- | (SEQ ID NO: 55) | -HPTNR- | (SEQ ID NO: 56) | -PHSKR- | (SEQ ID NO: 57) |
| -HHSKR- | (SEQ ID NO: 58) | -HPSKR- | (SEQ ID NO: 59) | -PHTKR- | (SEQ ID NO: 60) |
| -HHTKR- | (ΣEΘ ID NO: 61) | -HPTKR- | (SEQ ID NO: 62) | -PHSCK- | (SEQ ID NO: 63) |
| -HHSCK- | (SEQ ID NO: 64) | -HPSCK- | (SEQ ID NO: 65) | -PHTCK- | (SEQ ID NO: 66) |
| -HHTCK- | (SEQ ID NO: 67) | -HPTCK- | (SEQ ID NO: 68) | -PHSNK- | (SEQ ID NO: 69) |
| -HHSNK- | (SEQ ID NO: 70) | -HPSNK- | (SEQ ID NO: 71) | -PHTNK- | (SEQ ID NO: 72) |
| -HHTNK- | (SEQ ID NO: 73) | -HPTNK- | (SEQ ID NO: 74) | -PHSKK- | (SEQ ID NO: 75) |
| -HHSKK- | (SEQ ID NO: 76) | -HPSKK- | (SEQ ID NO: 77) | -PHTKK- | (SEQ ID NO: 78) |
| -HHTKK- | (SEQ ID NO: 79) | -HPTKK- | (SEQ ID NO: 80) | | |

In another embodiment of the present method, the anti-angiogenic peptide comprises, or preferably, consists of,

| | |
|---|---|
| X₁-X₂-X₃-X₄-X₅, | (SEQ ID NO: 81) |

wherein X₁ is an amino acid selected from the group consisting of proline, glycine, valine, histidine, isoleucine, phenylalanine, tyrosine, and tryptophan, and X₂ is an amino acid selected from the group consisting of histidine, proline, tyrosine, asparagine, glutamine, arginine, lysine, phenylalanine, and tryptophan, and X₃ is an amino acid selected from the group consisting of serine, threonine, alanine, tyrosine, leucine, histidine, asparagine, and glutamine, and X₄ is an amino acid selected from the group consisting of arginine, lysine, and histidine, and X₅ is an amino acid selected from the group consisting of asparagine, glutamine, serine, threonine, histidine, and tyrosine.

In another embodiment, the anti-angiogenic peptide has a Cys group substituting for one of the positions of SEQ ID NO:2. Again, the preferred substituent is PHSCN (SEQ ID NO:1). Other variants are pentapeptides or addition variants, as described above, but with one of the following core sequences: CHSRN (SEQ ID NO:82), PCSRN (SEQ ID NO:83), PHCRN (SEQ ID NO:84), and PHSRC (SEQ ID NO:85). The L-Cys in these peptides can be substituted by any sulfhydryl containing amino acid. Preferred examples are D-Cys, L- or D-homocysteine (Hcy) or D- or L-penicillamine. Penicillamine is also known as, 3-dimethylcysteine˜3-mercaptovaline; H-Pen-OH; 3,3-dimethylcysteine; and 2-amino-3-mercapto-3-methylbutanoic acid.

In yet another embodiment, the anti-angiogenic composition comprises, consists essentially of, or consists of, a pentapeptide or derivative having the amino acid sequence

| | |
|---|---|
| X₁-H-S-X₂-N, | (SEQ ID NO: 86) |

wherein X₁ is either proline, histidine, or is not an amino acid, and X₂ is L-cysteine, D-cysteine, homocysteine (Hcy), histidine, penicillamine or any other sulfhydryl containing amino acid.

A preferred embodiment of the peptide with SEQ ID NO:86 is one with the sequence PHSXN (SEQ ID NO:87), wherein X is L- or D-cysteine, L- or D-homocysteine, L- or D-penicillamine, any other amino acid with a sulfhydryl group, or L- or D-histidine.

The method of the present invention may employ a peptide, preferably a pentapeptide, but also a longer peptide up to about 30 residues, which comprises at least one stretch of the following tetrameric sequences:

| | | | | | |
|---|---|---|---|---|---|
| -PSCN- | (SEQ ID NO: 88) | -HSCN- | (SEQ ID NO: 89) | -HTCN- | (SEQ ID NO: 90) |
| -PTCN- | (SEQ ID NO: 91) | -HSCR- | (SEQ ID NO: 92) | -PSCR- | (SEQ ID NO: 93) |
| -HTCR- | (SEQ ID NO: 94) | -PTCR- | (SEQ ID NO: 95) | -HSCK- | (SEQ ID NO: 96) |
| -PSCK- | (SEQ ID NO: 97) | -HTCK- | (SEQ ID NO: 98) | -PTCK- | (SEQ ID NO: 99). |

A control peptide derivative, ATN-163, that is useful as a negative control for comparison testing, has the same amino acid composition as ATN-161, but the sequence is scrambled so that its sequence is His-Ser-Pro-Asn-Cys. ATN-163 is the capped form: Ac-HSPNC—NH₂).

As noted above, this invention includes use of peptides in which at least one amino acid residue and preferably, only one, has been removed and a different residue inserted in its place compared to the “base” sequence of PHSCN (SEQ ID NO:1). For a detailed description of protein chemistry and structure, see Schulz, G. E. et al., Principles of Protein Structure, Springer-Verlag, New York, 1979, and Creighton, T. E., Proteins: Structure and Molecular Principles, W.H. Freeman & Co., San Francisco, 1984, which are hereby incorporated by reference. One type of preferred substitution is a conservative substitutions, well known in the art, and generally considered to be exchanges within one of the following groups:

1. Small aliphatic, nonpolar or slightly polar residues: e.g., Ala, Ser, Thr, Gly;
2. Polar, negatively charged residues and their amides: e.g., Asp, Asn, Glu, Gln;
3. Polar, positively charged residues: e.g., His, Arg, Lys;
Pro, because of its unusual geometry, tightly constrains the chain.

Substantial changes in functional properties are made by selecting substitutions that are less conservative, such as between, rather than within, the above groups (or two other amino acid groups not shown above), which will differ more significantly in their effect on maintaining (a) the structure of the peptide backbone in the area of the substitution (b) the charge or hydrophobicity of the molecule at the target site, or (c) the bulk of the side chain. Most substitutions according to the present invention are those that do not produce radical changes in the characteristics of the peptide molecule. Even when it is difficult to predict the exact effect of a substitution in advance of doing so, one skilled in the art will appreciate that the effect can be evaluated by routine screening assays, preferably the biological assays described herein or others well known in the art of angiogenesis research. Modifications of peptide properties including redox or thermal stability, hydrophobicity, susceptibility to proteolytic degradation or the tendency to aggregate with carriers or into multimers fare assayed by methods well known to the ordinarily skilled artisan.

### Terminal Capping of Peptides

The peptide used herein is preferably capped at its N- and C-termini with an acyl (abbreviated “Ac”)- and an amido (abbreviated “Am”) group, respectively, for example acetyl (CH₃CO—) at the N terminus and amido (—NH₂) at the C terminus. (“Acetyl: is also abbreviated in some places herein as “Ac” when used in conjunction with an —NH₂ cap at the C-terminus.) A broad range of N-terminal capping functions, preferably in a linkage to the terminal amino group, is contemplated, for example:

formyl;

alkanoyl, having from 1 to 10 carbon atoms, such as acetyl, propionyl, butyryl;

alkenoyl, having from 1 to 10 carbon atoms, such as hex-3-enoyl;

alkynoyl, having from 1 to 10 carbon atoms, such as hex-5-ynoyl;

aroyl, such as benzoyl or 1-naphthoyl;

heteroaroyl, such as 3-pyrroyl or 4-quinoloyl;

alkylsulfonyl, such as methanesulfonyl;

arylsulfonyl, such as benzenesulfonyl or sulfanilyl;

heteroarylsulfonyl, such as pyridine-4-sulfonyl;

substituted alkanoyl, having from 1 to 10 carbon atoms, such as 4-aminobutyryl;

substituted alkenoyl, having from 1 to 10 carbon atoms, such as 6-hydroxy-hex-3-enoyl;

substituted alkynoyl, having from 1 to 10 carbon atoms, such as 3-hydroxy-hex-5-ynoyl;

substituted aroyl, such as 4-chlorobenzoyl or 8-hydroxy-naphth-2-oyl;

substituted heteroaroyl, such as 2,4-dioxo-1,2,3,4-tetrahydro-3-methyl-quinazolin-6-oyl;

substituted alkylsulfonyl, such as 2-aminoethanesulfonyl;

substituted arylsulfonyl, such as 5-dimethylamino-1-naphthalenesulfonyl;

substituted heteroarylsulfonyl, such as 1-methoxy-6-isoquinolinesulfonyl;

carbamoyl or thiocarbamoyl;

substituted carbamoyl(R′—NH—CO) or substituted thiocarbamoyl(R′—NH—CS) wherein R′ is alkyl, alkenyl, alkynyl, aryl, heteroaryl, substituted alkyl, substituted alkenyl, substituted alkynyl, substituted aryl, or substituted heteroaryl;

substituted carbamoyl(R′—NH—CO) and substituted thiocarbamoyl(R′—NH—CS) wherein R′ is alkanoyl, alkenoyl, alkynoyl, aroyl, heteroaroyl, substituted alkanoyl, substituted alkenoyl, substituted alkynoyl, substituted aroyl, or substituted heteroaroyl, all as above defined.

The C-terminal capping function can either be in an amide or ester bond with the terminal carboxyl. Capping functions that provide for an amide bond are designated as NR¹R² wherein R¹ and R² may be independently drawn from the following group:

hydrogen;

alkyl, preferably having from 1 to 10 carbon atoms, such as methyl, ethyl, isopropyl;

alkenyl, preferably having from 1 to 10 carbon atoms, such as prop-2-enyl;

alkynyl, preferably having from 1 to 10 carbon atoms, such as prop-2-ynyl;

substituted alkyl having from 1 to 10 carbon atoms, such as hydroxyalkyl, alkoxyalkyl, mercaptoalkyl, alkylthioalkyl, halogenoalkyl, cyanoalkyl, aminoalkyl, alkylaminoalkyl, dialkylaminoalkyl, alkanoylalkyl, carboxyalkyl, carbamoylalkyl;

substituted alkenyl having from 1 to 10 carbon atoms, such as hydroxyalkenyl, alkoxyalkenyl, mercaptoalkenyl, alkylthioalkenyl, halogenoalkenyl, cyanoalkenyl, aminoalkenyl, alkylaminoalkynyl, dialkylaminoalkynyl, alkanoylalkynyl, carboxyalkynyl, carbamoylalkynyl;

substituted alkynyl having from 1 to 10 carbon atoms, such as hydroxyalkynyl, alkoxyalkynyl, mercaptoalkynyl, alkylthioalkynyl, halogenoalkynyl, cyanoalkynyl, aminoalkynyl, alkylaminoalkynyl, dialkylaminoalkynyl, alkanoylalkynyl, carboxyalkynyl, carbamoylalkynyl;

aroylalkyl having up to 10 carbon atoms, such as phenacyl or 2-benzoylethyl;

aryl, such as phenyl or 1-naphthyl;

heteroaryl, such as 4-quinolyl;

alkanoyl having from 1 to 10 carbon atoms, such as acetyl or butyryl;

aroyl, such as benzoyl;

heteroaroyl, such as 3-quinoloyl;

OR′ or NR′R″ where R′ and R″ are independently hydrogen, alkyl, aryl, heteroaryl, acyl, aroyl, sulfonyl, sulfinyl, or SO₂—R′″ or SO—R′″ where R′″ is substituted or unsubstituted alkyl, aryl, heteroaryl, alkenyl, or alkynyl.

Capping functions that provide for an ester bond are designated as OR, wherein R may be: alkoxy; aryloxy; heteroaryloxy; aralkyloxy; heteroaralkyloxy; substituted alkoxy; substituted aryloxy; substituted heteroaryloxy; substituted aralkyloxy; or substituted heteroaralkyloxy.

Either the N-terminal or the C-terminal capping function, or both, may be of such structure that the capped molecule functions as a prodrug (a pharmacologically inactive derivative of the parent drug molecule) that undergoes spontaneous or enzymatic transformation within the body in order to release the active drug and that has improved delivery properties over the parent drug molecule (Bundgaard H, Ed: Design of Prodrugs, Elsevier, Amsterdam, 1985).

Judicious choice of capping groups allows the addition of other activities to the peptide. For example, the presence of a sulfhydryl group linked to the N- or C-terminal cap will permit conjugation of the derivatized peptide to other molecules.

### Peptidomimetics and Chemical Derivatives of the Peptide

A preferred type of chemical derivative of the peptides described herein is a peptidomimetic compound which mimics the biological effects of PHSCN (SEQ ID NO:1). A peptidomimetic agent may be an unnatural peptide or a non-peptide agent that recreates the stereospatial properties of the binding elements of the peptide which it mimics, such that it has the binding activity or biological activity of the original peptide. Similar to biologically active peptides, a peptidomimetic will have a binding face (which interacts with any ligand to which the natural peptide binds) and a non-binding face. The non-binding face of a peptidomimetic will contain functional groups which can be modified by various therapeutic moieties without modifying the binding face of the peptidomimetic One embodiment of a peptidomimetic would contain an aniline on the non-binding face of the molecule. The NH₂-group of an aniline has a pKa˜4.5 and could therefore be modified by any NH₂-selective reagent without modifying any NH₂ functional groups on the binding face of the peptidomimetic. Other peptidomimetics may have no NH₂ functional groups on their binding face and therefore, any NH₂, without regard for pKₐ could be displayed on the non-binding face as a site for conjugation. In addition other modifiable functional groups, such as —SH and —COOH could be incorporated into the non-binding face of a peptidomimetic as a site of conjugation. A therapeutic moiety could also be directly incorporated during the synthesis of a peptidomimetic and preferentially be displayed on the non-binding face of the molecule.

This invention also includes compounds that retain partial peptide characteristics. For example, any proteolytically unstable bond within a peptide of the invention could be selectively replaced by a non-peptidic element such as an isostere (N-methylation; D-amino acid) or a reduced peptide bond while the rest of the molecule retains its peptide nature.

Peptidomimetic compounds, either agonists, substrates or inhibitors, have been described for a number of bioactive peptides such as opioid peptides, VIP, thrombin, HIV protease, etc. Methods for designing and preparing peptidomimetic compounds are known in the art (Hruby, V. J., Biopolymers 33:1073-1082 (1993); Wiley, R. A. et al., Med. Res. Rev. 13:327-384 (1993); Moore et al., Adv. in Pharmacol 33:91-141 (1995); Giannis et al., Adv. in Drug Res. 29:1-78 (1997), which references are incorporated by reference in their entirety). These methods are used to make peptidomimetics that possess at least the binding capacity and specificity of the PHSCN (SEQ ID NO:1) peptide and preferably also possess the biological activity. Knowledge of peptide chemistry and general organic chemistry available to those skilled in the art are sufficient, in view of the present disclosure, for designing and synthesizing such compounds.

For example, such peptidomimetics may be identified by inspection of the cystallographically-derived three-dimensional structure of a peptide of the invention either free or bound in complex with a ligand such as an integrin. Alternatively, the structure of the natural peptide of the invention bound to its ligand can be gained by the techniques of nuclear magnetic resonance spectroscopy. The better knowledge of the stereochemistry of the interaction of the peptide with its ligand or receptor will permit the rational design of such peptidomimetic agents. The structure of a peptide or protein of the invention in the absence of ligand could also provide a scaffold for the design of mimetic molecules.

A preferred chemical derivative/mimetic of the peptides described above is a cyclic peptide with some or most of the same amino acid residues but which is further stabilized by nonpeptidic bonds. Methods for making and using such compounds are described, for example, in U.S. Pat. No. 5,192,746 to Lob1, et al., U.S. Pat. No. 5,169,862 to Burke, Jr., et al., U.S. Pat. No. 5,539,085 to Bischoff, et al., U.S. Pat. No. 5,576,423 to Aversa, et al., U.S. Pat. No. 5,051,448 to Shashoua, and U.S. Pat. No. 5,559,103 to Gaeta, et al., all of which are hereby incorporated by reference. Synthesis of nonpeptidic compounds that mimic peptides is also known in the art. Eldred, et al., 1994, J. Med. Chem. 37:3882, describes nonpeptidic antagonists that mimic the sequence Arg-Gly-Asp (RGD). Ku et al., 1995, J. Med. Chem. 38:9, further elucidates synthesis of a series of such compounds.

### Production of Peptides and Derivatives

The peptides of the invention may be prepared using recombinant DNA technology. However, given their length, they are preferably prepared using solid-phase synthesis, such as that generally described by Merrifield, J. Amer. Chem. Soc., 85:2149-54 (1963), although other equivalent chemical syntheses known in the art are also useful. Solid-phase peptide synthesis may be initiated from the C-terminus of the peptide by coupling a protected α-amino acid to a suitable resin. Such a starting material can be prepared by attaching an α-amino-protected amino acid by an ester linkage to a chloromethylated resin or to a hydroxymethyl resin, or by an amide bond to a BHA resin or MBHA resin. Such methods, well-known in the art, are disclosed, for example, in U.S. Pat. No. 5,994,309 which is incorporated by reference in its entirety.

### In Vitro Testing of Compositions

General descriptions of methods (in vitro, ex vivo, in vivo) used for study of angiogenesis, albeit focusing on tumors but which are generally applicable, appear in Vermeulen P B et al., 1996, Eur J Cancer 32A:2474-84 and Vermeulen P B et al., 2002, Eur J Cancer 38:1564-79.

### In Vitro or Ex Vivo Testing of Compositions

A. Microvessel Density (MVD) Analysis

This is a well known histologic method described in the Examples below. For a general description, see, for example, Gasparini, G et al., 1994, J. Clin. Oncol. 12:454-466; Axelsson K et al., 1995, J Natl Cancer Inst. 87:997-1008; Hansen, S. et al., 2003, Brit J Cancer 88:102-108; Offersen B V et al., 2003, Eur J Cancer 39:881-90; Amis, S J et al., 2005, Int J Gynecol Cancer. 15:58-65.

MVD in paraffin sections of tissue samples can be correlated with microvessel counts from frozen sections. Any acceptable endothelial cell marker (typically mAbs) can be used, including anti-CD31, a pan-endothelial marker, CD105 (ligand for TGFβ) or human von Willebrand factor. MVD is typically performed in neovascular hotspots, for example, using a Quantimet 500+ Image Analyzer. The highest vessel density (HVD) and average vessel density (AVD) of a desired number of fields, e.g., three, are recorded. Amis et al., supra, (studying ovarian tumors and cysts) and comparing fixed and frozen tissue found a strong correlation between the HVD and AVD at magnifications tested (×200 and ×400). The good correlation between MVD in fixed and frozen sections suggests that such observations represent a true reflection of angiogenesis in both physiologic and pathologic states. In fixed tissue, the HVD and AVD were significantly greater in the group containing functional ovarian cysts which showed even more development of microcirculation than in tumors.

Chantrain C F et al., 2003, J Histochem Cytochem. 51:151-58 describe various strengths and weaknesses of assessing tissue vascularization using immunohistochemical techniques for MVD that include subjective factors. Objective criteria were introduced with imaging analysis software and a high-resolution slide scanner for measurement of CD31-immunostained “endothelial area” in whole sections of murine or xenografted human tumors. The use of the criteria on images of entire (tumor) section acquired with the slide scanner constitutes a rapid method to quantify vascularization. Compared with “hot spot” and the “random fields” methods, endothelial area measurements obtained with a “whole section scanning” method are more reproducible. Another computerized image analysis system has recently been developed by Barbareschi, M et al. (1995, in press).

B. Assay for Endothelial Cell Migration

For EC migration, transwells are coated with type I collagen (50 μg/mL) by adding 200 μL of the collagen solution per transwell, then incubating overnight at 37° C. The transwells are assembled in a 24-well plate and a chemoattractant (e.g., FGF-2) is added to the bottom chamber in a total volume of 0.8 mL media. ECs, such as human umbilical vein endothelial cells (HUVEC), which have been detached from monolayer culture using trypsin, are diluted to a final concentration of about 10⁶ cells/mL with serum-free media and 0.2 mL of this cell suspension is added to the upper chamber of each transwell. Inhibitors to be tested are added to both the upper and lower chambers, and the migration is allowed to proceed for 5 hrs in a humidified atmosphere at 37° C. The transwells are removed from the plate stained using DiffQuik®. Cells which did not migrate are removed from the upper chamber by scraping with a cotton swab and the membranes are detached, mounted on slides, and counted under a high-power field (400×) to determine the number of cells migrated.

C. Tube-Formation Assays of Anti-Angiogenic Activity

The compounds of this invention are tested for their anti-angiogenic activity in one of two different assay systems in vitro.

Endothelial cells, for example, human umbilical vein endothelial cells (HUVEC) or human microvascular endothelial cells (HMVEC) which can be prepared or obtained commercially, are mixed at a concentration of 2×10⁵ cells/mL with fibrinogen (5 mg/mL in phosphate buffered saline (PBS) in a 1:1 (v/v) ratio. Thrombin is added (5 units/mL final concentration) and the mixture is immediately transferred to a 24-well plate (0.5 mL per well). The fibrin gel is allowed to form and then VEGF and bFGF are added to the wells (each at 5 ng/mL final concentration) along with the test compound. The cells are incubated at 37° C. in 5% CO₂ for 4 days at which time the cells in each well are counted and classified as either rounded, elongated with no branches, elongated with one branch, or elongated with 2 or more branches. Results are expressed as the average of 5 different wells for each concentration of compound. Typically, in the presence of angiogenic inhibitors, cells remain either rounded or form undifferentiated tubes (e.g. 0 or 1 branch). This assay is recognized in the art to be predictive of angiogenic (or anti-angiogenic) efficacy in vivo (Min, H Y et al., Cancer Res. 56: 2428-2433 (1996)).

In an alternate assay, endothelial cell tube formation is observed when endothelial cells are cultured on Matrigel® (Schnaper et al., J. Cell. Physiol. 165:107-118 1995). Endothelial cells (1×10⁴ cells/well) are transferred onto Matrigel®-coated 24-well plates, and tube formation is quantitated after 48 hrs. Inhibitors are tested by adding them either at the same time as the endothelial cells or at various time points thereafter. Tube formation can also be stimulated by adding (a) angiogenic growth factors such as bFGF or VEGF, (b) differentiation stimulating agents (e.g., PMA) or (c) a combination of these.

This assay models angiogenesis by presenting to the endothelial cells a particular type of basement membrane, namely the layer of matrix which migrating and differentiating endothelial cells might be expected to first encounter. In addition to bound growth factors, the matrix components found in Matrigel® (and in basement membranes in situ) or proteolytic products thereof may also be stimulatory for endothelial cell tube formation which makes this model complementary to the fibrin gel angiogenesis model previously described (Blood et al., Biochim. Biophys. Acta 1032:89-118, 1990; Odedra et al., Pharmac. Ther. 49:111-124, 1991). The compounds of this invention inhibit endothelial cell tube formation in both assays, which suggests that the compounds will also have anti-angiogenic activity.

### In Vivo Testing of Peptides

Certain general methods for evaluating angiogenesis are well known in the art and are described briefly below.

A. Corneal Angiogenesis Model

The protocol used is essentially identical to that described by Volpert et al. (J. Clin. Invest. 98:671-679 (1996)). Briefly, female Fischer rats (120-140 gms) are anesthetized and pellets (5 μl) comprised of Hydron®, bFGF (150 nM), and the compounds to be tested are implanted into tiny incisions made in the cornea 1.0-1.5 mm from the limbus. Neovascularization is assessed at 5 and 7 days after implantation. On day 7, animals are anesthetized and infused with a dye such as colloidal carbon to stain the vessels. The animals are then euthanized, the corneas fixed with formalin, and the corneas flattened and photographed to assess the degree of neovascularization. Neovessels may be quantitated by imaging the total vessel area or length or simply by counting vessels.

B. Matrigel® Plug Assay

This assay is performed essentially as described by Passaniti et al. (Lab Invest. 67:519-528 (1992). Ice-cold Matrigel® (e.g., 500 μL) (Collaborative Biomedical Products, Inc., Bedford, Mass.) is mixed with heparin (e.g., 50 μg/ml), FGF-2 (e.g., 400 ng/ml) and the compound to be tested. In some assays, bFGF may be substituted with tumor cells as the angiogenic stimulus. The Matrigel® mixture is injected subcutaneously into 4-8 week-old athymic nude mice at sites near the abdominal midline, preferably 3 injections per mouse. The injected Matrigel® forms a palpable solid gel. Injection sites are chosen such that each animal receives a positive control plug (such as FGF-2+heparin), a negative control plug (e.g., buffer+heparin) and a plug that includes the compound being tested for its effect on angiogenesis, e.g., (FGF-2+heparin+compound). All treatments are preferably run in triplicate. Animals are sacrificed by cervical dislocation at about 7 days post injection or another time that may be optimal for observing angiogenesis. The mouse skin is detached along the abdominal midline, and the Matrigel® plugs are recovered and scanned immediately at high resolution. Plugs are then dispersed in water and incubated at 37° C. overnight. Hemoglobin (Hb) levels are determined using Drabkin's solution (e.g., obtained from Sigma) according to the manufacturers' instructions. The amount of Hb in the plug is an indirect measure of angiogenesis as it reflects the amount of blood in the sample. In addition, or alternatively, animals may be injected prior to sacrifice with a 0.1 ml buffer (preferably PBS) containing a high molecular weight dextran to which is conjugated a fluorophore. The amount of fluorescence in the dispersed plug, determined fluorimetrically, also serves as a measure of angiogenesis in the plug. Staining with mAb anti-CD31 (CD31 is “platelet-endothelial cell adhesion molecule or PECAM”) may also be used to confirm neovessel formation and microvessel density in the plugs.

C. Chick Chorioallantoic Membrane (CAM) Angiogenesis Assay

This assay is performed essentially as described by Nguyen et al. (Microvascular Res. 47:31-40 (1994)). A mesh containing either angiogenic factors (bFGF) or tumor cells plus inhibitors is placed onto the CAM of an 8-day old chick embryo and the CAM observed for 3-9 days after implantation of the sample. Angiogenesis is quantitated by determining the percentage of squares in the mesh which contain blood vessels.

### A Murine Model of Crohn's Disease

The preferred, and widely used, model for testing is the IL-10-deficient (KO) mouse (on a C57BL/10 strain background). As described in the Examples, colonies of C57BL/10 IL-10 KO mice are bred and used. These mice consistently develop colitis at 10-12 weeks of age when transported from the ultra-barrier facility to conventional housing. These mice are used to test the effect of a candidate anti-angiogenic agent on pathogenesis and treatment of established disease.

### Pharmaceutical Compositions and Therapeutic Methods

The preferred animal subject of the present invention is a mammal. The invention is particularly useful in the treatment of human subjects. By the term “treating” is intended the administering to subjects of a pharmaceutical composition comprising the peptides described herein

The term “systemic administration” refers to administration of a peptides or derivative described herein, in a manner that results in the introduction of the composition into the subject's circulatory system or otherwise permits its spread throughout the body, such as intravenous (i.v.) injection or infusion. “Regional” administration refers to administration into a specific, and somewhat more limited, anatomical space, such as intraperitoneal, intrathecal, subdural, or to a specific organ. Examples include intravaginal, intrapenile, intranasal, intrabronchial (or lung instillation), intracranial, intra-aural or intraocular. The term “local administration” refers to administration of a composition or drug into a limited, or circumscribed, anatomic space, such as intratumoral injection into a tumor mass, subcutaneous (s.c.) injections, intramuscular (i.m.) injections. One of skill in the art would understand that local administration or regional administration often also result in entry of a composition into the circulatory system, i.e., so that s.c. or i.m. are also routes for systemic administration.

Injectables or infusible preparations can be prepared in conventional forms, either as solutions or suspensions, solid forms suitable for solution or suspension in liquid prior to injection or infusion, or as emulsions. Though the preferred routes of administration are systemic, such as i.v., the pharmaceutical composition may be administered topically or transdermally, e.g., as an ointment, cream or gel; orally; rectally; e.g., as a suppository.

For topical application, the compound may be incorporated into topically applied vehicles such as a salve or ointment. The carrier for the active ingredient may be either in sprayable or nonsprayable form. Non-sprayable forms can be semi-solid or solid forms comprising a carrier indigenous to topical application and having a dynamic viscosity preferably greater than that of water.

Other pharmaceutically acceptable carriers for the peptide compositions are liposomes, pharmaceutical compositions in which the active protein is contained either dispersed or variously present in corpuscles consisting of aqueous concentric layers adherent to lipidic layers. The active polypeptide or peptide, or the nucleic acid is preferably present in the aqueous layer and in the lipidic layer, inside or outside, or, in any event, in the non-homogeneous system generally known as a liposomic suspension. The hydrophobic layer, or lipidic layer, generally, but not exclusively, comprises phospholipids such as lecithin and sphingomyelin, steroids such as cholesterol, more or less ionic surface active substances such as dicetylphosphate, stearylamine or phosphatidic acid, and/or other materials of a hydrophobic nature. Those skilled in the art will appreciate other suitable embodiments of the present liposomal formulations.

The therapeutic dosage administered is an amount which is therapeutically effective, as is known to or readily ascertainable by those skilled in the art. The dose is also dependent upon the age, health, and weight of the recipient, kind of concurrent treatment(s), if any, the frequency of treatment, and the nature of the effect desired, such as, for example, anti-inflammatory effects or anti-bacterial effect.

The pharmaceutical compositions of the present invention wherein the peptide is combined with pharmaceutically acceptable excipient or carrier, may be administered by any means that achieve their intended purpose. Amounts and regimens for the administration of can be determined readily by those with ordinary skill in the clinical art of treating any of the particular diseases. Preferred amounts are described below.

Pharmaceutical compositions within the scope of this invention include all compositions wherein the peptide is contained in an amount effective to achieve its intended purpose. While individual needs vary, determination of optimal ranges of effective amounts of each component is within the skill of the art. As noted below, typical dosages comprise between about 1 μg/kg/body wt and about 100 mg/kg/body wt, though more preferred dosages are described for certain particular uses, below.

As stated above, in addition to the pharmacologically active peptide, the pharmaceutical compositions may contain suitable pharmaceutically acceptable carriers comprising excipients and auxiliaries which facilitate processing of the active compounds into preparations which can be used pharmaceutically as is well known in the art. Suitable solutions for administration by injection or orally, may contain from about 0.01 to 99 percent, active compound(s) together with the excipient.

The pharmaceutical compositions of the present invention are manufactured in a manner which is itself known, for example, by means of conventional mixing, granulating, dissolving, or lyophilizing processes. Suitable excipients may include fillers binders, disintegrating agents, auxiliaries and stabilizers, all of which are known in the art. Suitable formulations for parenteral administration include aqueous solutions of the proteins in water-soluble form, for example, water-soluble salts. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions that may contain substances which increase the viscosity of the suspension.

The pharmaceutical formulation for systemic administration according to the invention may be formulated for enteral, parenteral or topical administration, and all three types of formulation may be used simultaneously to achieve systemic administration of the active ingredient.

### Treatment/Amelioration of Inflammatory Bowel Disease and Related Conditions

Doses of the present pharmaceutical compositions preferably include pharmaceutical dosage units comprising an effective amount of the peptide. Dosage unit form refers to physically discrete units suited as unitary dosages for a mammalian subject; each unit contains a predetermined quantity of active material calculated to produce the desired therapeutic effect, in association with the required pharmaceutical carrier. The specification for the dosage unit forms of the invention are dictated by and directly dependent on (a) the characteristics of the active material and the particular therapeutic effect to be achieved, and (b) the limitations inherent in the art of compounding such an active compound for the treatment of, and sensitivity of, individual subjects

By an effective amount is meant an amount sufficient to achieve a steady state concentration in vivo which results in a measurable reduction in any relevant parameter of disease which are well-known in the art, and, for the animal model of CrD, are exemplified below. See also, Scheinin et al., supra, and reference cited therein. This may include any accepted index of inflammatory reactivity, or a measurable prolongation of disease-free interval or of survival.

In one embodiment, an effective dose is preferably 10-fold and more preferably 100-fold higher than the 50% effective dose (ED₅₀) of the compound in an in vivo assay as described herein.

The amount of active compound to be administered depends on the precise peptide or derivative selected, the route of administration, the health and weight of the recipient, the existence of other concurrent treatment, if any, the frequency of treatment, the nature of the effect desired, for example, inhibition of tumor metastasis, and the judgment of the skilled practitioner.

A preferred dose for treating a subject, preferably mammalian, more preferably human, CrD or other IBD is an amount of up to about 100 milligrams of active peptide-based compound per kilogram of body weight. A typical single dosage of the peptide or peptidomimetic is between about 1 μg and about 100 mg/kg body weight. A total daily dosage in the range of about 0.1 milligrams to about 7 grams is preferred for intravenous administration. The foregoing ranges are, however, suggestive, as the number of variables in an individual treatment regime is large, and considerable excursions from these preferred values are expected.

Effective doses and optimal dose ranges may be determined based on in vitro or animal studies using the methods described herein.

Therapeutic compositions for treating IBD, particularly CrD may comprise, in addition to the peptide, one or more additional anti-inflammatory agents or other medicaments that treat additional symptoms for which the target patients are at risk

Having now generally described the invention, the same will be more readily understood through reference to the following examples which are provided by way of illustration, and are not intended to be limiting of the present invention, unless specified.

### Example I

### ATN-151 Reduced Colitis in IL-10-Deficient Mice with Established Inflammatory Bowel Disease

Colonies of C57BL/10 IL-10-deficient mice were routinely bred at the Case Western Reserve University Animal Research Center for studies of early and late colitis. These mice consistently develop colitis at 10-12 weeks of age when transported from the ultra-barrier facility to conventional housing. These mice were used to test the effect of ATN 161 (Ac-PHSCN—NH₂) on both the development and treatment of established disease.

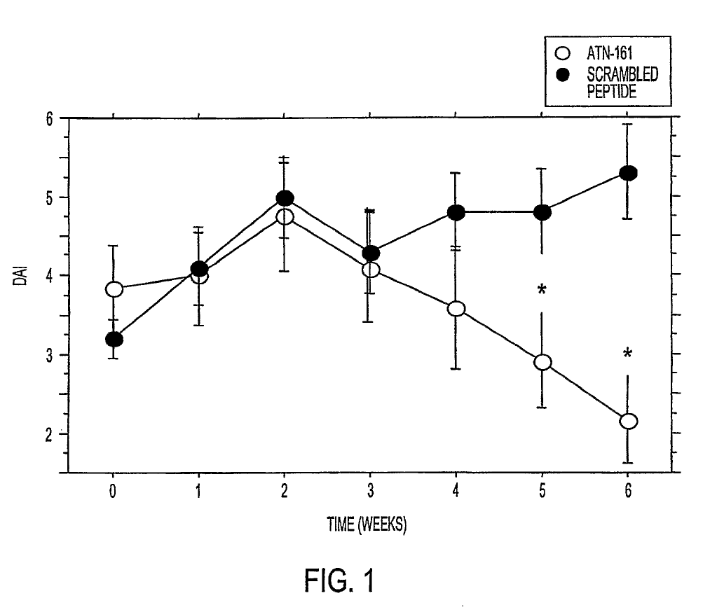

ATN-161 had no effect on the development of colitis in this model when treatment was initiated at the time that the mice were moved to conventional housing. However, ATN-161 consistently reduced colitis in mice with established disease (FIG. 1) an effect that was observed after approximately four weeks of treatment. A scrambled peptide version of ATN-161 (ATN-163; Ac-HSPNC—NH₂) was used as a control and had no effect on established colitis in this model. ATN-163 and vehicle controls were indistinguishable in these studies (data not shown).

### Example II

### ATN-161 Treated Animals Expressed Lower Histologic Scores and Produced Less IL-6 and IL-12 in Organ Culture

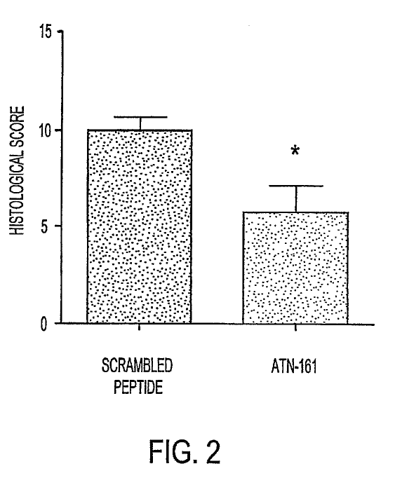

Animals were euthanized at the end of Week 6 and the colon removed for histologic analysis. The ATN-161 treated group had a significantly lower histologic score than the ATN-163 treated group (FIG. 2).

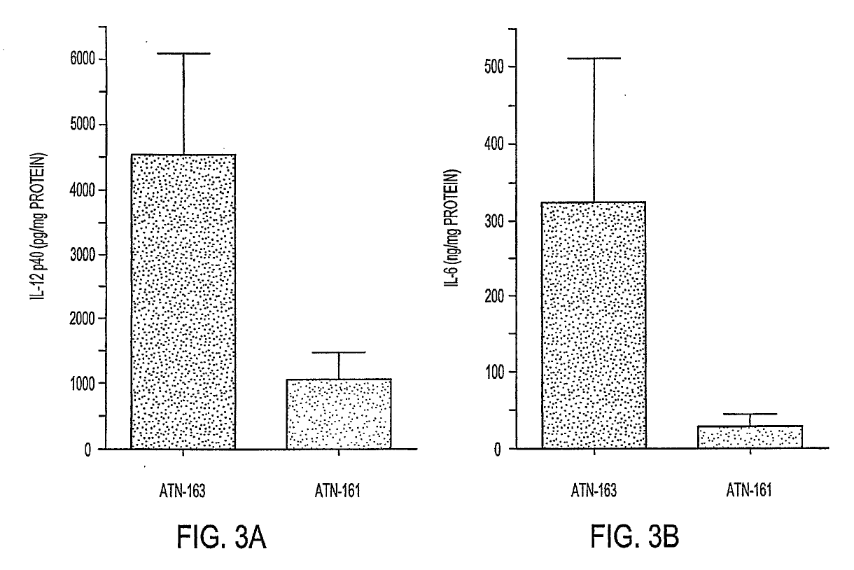

Biopsies were also cultured in vitro and the supernatants analyzed for IL-6 and IL-12 expression as previously described (Spencer D M et al., 2002, Gastroenterology 122:94-105). IL-6 and IL-12 are inflammatory cytokines that have been correlated with the pathogenesis of colitis (Mahida Y R, 2000, Inflamm Bowel Dis 6:21-33). Colon fragments from ATN-161 treated animals expressed significantly lower levels of IL-6 and IL-12 in organ culture than ATN-163 treated controls (FIG. 3).

The effect of ATN-161 on IL-6 and IL-12 expression was also evaluated in vitro using lamina propria mononuclear cell (LPMC) extracts and splenocytes. ATN-161 had no effect on IL-6 and IL-12 production in these cells, suggesting that the effect of ATN-161 is due to a general decrease of intestinal microvasculature, rather than because of a direct effect on LPMC.

### Example III

### Reduced Angiogenesis in ATN-161-Treated Mice

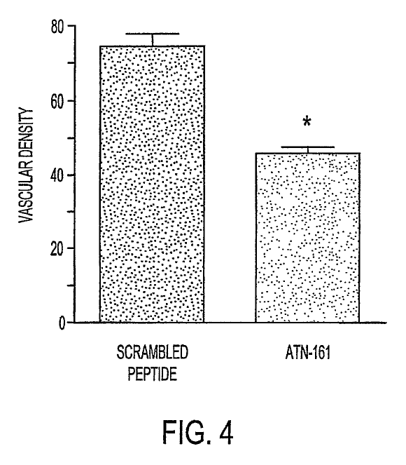

Microvessel density as a measure of angiogenesis was determined in zinc fixed colon sections. Colon fragments from ATN-161 treated mice had approximately 40% less microvessels than the ATN-163 treated controls demonstrating an inhibition of angiogenesis in this model (FIG. 4).

ATN-161 in the IL-10 knockout mouse model of CrD shows activity as measured by the Disease Activity Index and histologic grading of colon tissue. This activity is associated with decreases in IL-6 and IL-12 production by cultured colon tissue and decreases in microvessel density. These finding indicate that the anti-angiogenesis agent ATN-161 (a capped pentapeptide) is useful for treating human CrD.

### DISCUSSION

The present inventors are the first to have shown the therapeutic benefits in IBD of directly inhibiting angiogenesis. Without wishing to be bound or limited by any mechanistic explanation, it is clear to the inventors that certain advantages can result from anti-angiogenic therapy of IBD in light of what is known in other areas of chronic inflammation (see, for example, Griffioen A W et al., 2000, Pharmacol Rev 52:237-268). First, suppression of blood vessel growth diminishes nutrient supply to metabolically active cells in inflamed tissue. Second, by preventing blood vessel formation, the entry of inflammatory cells into tissues is attenuated. Third, inhibiting angiogenesis blocks EC activation and production of cytokines, chemokines and matrix metalloproteinases (MMPs). Angiogenesis may be interfered at several “levels” including intervening in EC growth, adhesion and migration, and inhibiting MMP activity (Griffioen et al., supra). Considerable experimental evidence in animal models has suggested that blocking angiogenesis improves inflammation. TNF-α blockade, which inhibits production of VEGF and bFGF, induces remission in CrD patients (Di Sabatino A et al., 2004, Aliment. Pharmacol. Ther. 19:1019-24). Thalidomide, another drug effective in CrD, acts via direct inhibition of TNF-α production (Ehrenpreis E D et al., 1999, Gastroenterology 117:1271-77) but can also inhibit angiogenesis. A recent report shows that thalidomide administration to patients with active CrD associated with intestinal bleeding promoted clinical improvement and stopped the bleeding (Bauditz J et al., 2004, Gut 53:609-12).

These clinical observations support the present inventors discovery and, in view of the results disclosed herein, help weave a mechanistic explanation that links inflammation, increased gut blood flow, and the therapeutic benefit of inhibiting excessive mucosal vascularization.

All references cited above, and references cited in those references, are incorporated by reference in their entirety (whether or not expressly incorporated above).

Having now fully described this invention, it will be appreciated by those skilled in the art that the same can be performed within a wide range of equivalent parameters, concentrations, and conditions without departing from the spirit and scope of the invention and without undue experimentation.

1. A method for decreasing the magnitude of intestinal inflammation or inflammatory infiltrate in bowel tissue, lowering systemic or gut-associated levels of a proinflammatory cytokine, or treating an inflammatory bowel disease in a subject with an inflammatory bowel disease, comprising, administering to a subject in need of such decreasing, lowering, reducing or treating an effective amount of a pharmaceutical composition that comprises
(a) a compound that inhibits angiogenesis; and
(b) a pharmaceutically acceptable carrier or excipient;
thereby decreasing said inflammation or infiltrate.

2. (canceled)

3. A method for reducing microvessel density, as determined in fixed bowel tissue sections, from a biopsy obtained from a subject with an inflammatory bowel disease, comprising
(a) administering to a subject in need of such treatment an effective amount of a pharmaceutical composition that comprises
(i) a compound that inhibits angiogenesis; and
(ii) a pharmaceutically acceptable carrier or excipient;
(b) obtaining a bowel biopsy from said subject, and
(c) determining the microvessel density in said biopsy,
wherein the administering of said compound results in a lower microvessel density compared to the microvessel density before receipt of the compound by the subject.

4. (canceled)

5. The method of claim 1, wherein the compound is a peptide of 5 to about 30 amino acid residues which comprises the amino acid sequence

| | |
|---|---|
| Xaa₁-Xaa₂-Xaa₃-Xaa₄-Xaa₅, | (SEQ ID NO: 81) |

wherein
Xaa₁ is Pro, Gly, Val, His, Iso, Phe, Tyr, or Trp;
Xaa₂ is His, Pro, Tyr, Asn, Glu, Arg, Lys, Phe, or Trp;
Xaa₃ is Ser, Thr, Ala, Tyr, Leu, His, Asn, or Glu;
Xaa₄ is L- or D-Cys, L- or D-Hcy, L- or D-penicillamine, any other amino acid having a —SH group, or L- or D-His;
Xaa₅ is Asn, Glu, Ser, Thr, His, or Tyr,
or a N- and C-terminally capped derivative of said peptide.

6. The method of claim 5 wherein the peptide has the amino acid sequence

| | |
|---|---|
| Xaa₁-His-Ser-Xaa₂-Asn, | (SEQ ID NO: 86) |

wherein Xaa₁ is Pro, His, or is not an amino acid, and Xaa₂ is L- or D-Cys, L- or D-Hcy, L- or D-penicillamine, or L- or D-His.

7. The method of claim 6 wherein the peptide has the amino acid sequence

| | |
|---|---|
| Pro-His-Ser-Xaa-Asn, | (SEQ ID NO: 87) |

wherein X is L- or D-Cys, L- or D-Hcy, L- or H-penicillamine, any other amino acid having a —SH group, or L- or D-His.

8. The method of claim 7 wherein the peptide has the amino acid sequence

| | |
|---|---|
| Pro-His-Ser-Cys-Asn. | (SEQ ID NO: 1) |

9. The method of claim 1 wherein the antiangiogenic compounds is a pentapeptide with the amino acid sequence

| | |
|---|---|
| Pro-His-Ser-Cys-Asn. | (SEQ ID NO: 1) |

10. The method of claim 5 wherein the peptide is N-terminally capped with an acetyl group and is C-terminally capped with an amino group.

11. The method of claim 1 wherein the subject is a human.

12. The method of claim 1 wherein said inflammatory bowel disease is Crohn's disease.

13-28. (canceled)

29. The method of claim 3 wherein the subject is a human.

30. The method of claim 3, wherein the compound is a peptide of 5 to about 30 amino acid residues which comprises the amino acid sequence

| | |
|---|---|
| Xaa₁-Xaa₂-Xaa₃-Xaa₄-Xaa₅, | (SEQ ID NO: 81) |

wherein
Xaa₁ is Pro, Gly, Val, His, Iso, Phe, Tyr, or Trp;
Xaa₂ is His, Pro, Tyr, Asn, Glu, Arg, Lys, Phe, or Trp;
Xaa₃ is Ser, Thr, Ala, Tyr, Leu, His, Asn, or Glu;
Xaa₄ is L- or D-Cys, L- or D-Hcy, L- or D-penicillamine, any other amino acid having a —SH group, or L- or D-His;
Xaa₅ is Asn, Glu, Ser, Thr, His, or Tyr,
or a N- and C-terminally capped derivative of said peptide.

31. The method of claim 30 wherein the peptide has the amino acid sequence

| | |
|---|---|
| Xaa₁-His-Ser-Xaa₂-Asn, | (SEQ ID NO: 86) |

wherein Xaa₁ is Pro, His, or is not an amino acid, and Xaa₂ is L- or D-Cys, L- or D-Hcy, L- or D-penicillamine, or L- or D-His.

32. The method of claim 31 wherein the peptide has the amino acid sequence

| | |
|---|---|
| Pro-His-Ser-Xaa-Asn, | (SEQ ID NO: 87) |

wherein X is L- or D-Cys, L- or D-Hcy, L- or H-penicillamine, any other amino acid having a —SH group, or L- or D-His.

33. The method of claim 32 wherein the peptide has the amino acid sequence

| | |
|---|---|
| Pro-His-Ser-Cys-Asn. | (SEQ ID NO: 1) |

34. The method of claim 3 wherein the antiangiogenic compounds is a pentapeptide with the amino acid sequence

| | |
|---|---|
| Pro-His-Ser-Cys-Asn. | (SEQ ID NO: 1) |

35. The method of claim 3 wherein said inflammatory bowel disease is Crohn's disease.


SEQUENCE LISTING (99 sequences)

SEQ ID NO: 1 - protein, 5 aa, Artificial Sequence
  Synthetic
PHSCN

SEQ ID NO: 2 - protein, 5 aa, Artificial Sequence
  Synthetic
PHSRN

SEQ ID NO: 3 - protein, 6 aa, Artificial Sequence
  Synthetic
PHSCNS

SEQ ID NO: 4 - protein, 7 aa, Artificial Sequence
  Synthetic
PHSCNSI

SEQ ID NO: 5 - protein, 8 aa, Artificial Sequence
  Synthetic
PHSCNSIT

SEQ ID NO: 6 - protein, 9 aa, Artificial Sequence
  Synthetic
PHSCNSITL

SEQ ID NO: 7 - protein, 10 aa, Artificial Sequence
  Synthetic
PHSCNSITLT

SEQ ID NO: 8 - protein, 11 aa, Artificial Sequence
  Synthetic
PHSCNSITLTN

SEQ ID NO: 9 - protein, 12 aa, Artificial Sequence
  Synthetic
PHSCNSITLTNL

SEQ ID NO: 10 - protein, 13 aa, Artificial Sequence
  Synthetic
PHSCNSITLTNLT

SEQ ID NO: 11 - protein, 14 aa, Artificial Sequence
  Synthetic
PHSCNSITLTNLTP

SEQ ID NO: 12 - protein, 15 aa, Artificial Sequence
  Synthetic
PHSCNSITLTNLTPG

SEQ ID NO: 13 - protein, 17 aa, Artificial Sequence
  Synthetic
EHFSGRPREDRVPHSCN

SEQ ID NO: 14 - protein, 18 aa, Artificial Sequence
  Synthetic
PEHFSGRPREDRVPHSCN

SEQ ID NO: 15 - protein, 16 aa, Artificial Sequence
  Synthetic
HFSGRPREDRVPHSCN

SEQ ID NO: 16 - protein, 15 aa, Artificial Sequence
  Synthetic
FSGRPREDRVPHSCN

SEQ ID NO: 17 - protein, 14 aa, Artificial Sequence
  Synthetic
SGRPREDRVPHSCN

SEQ ID NO: 18 - protein, 13 aa, Artificial Sequence
  Synthetic
GRPREDRVPHSCN

SEQ ID NO: 19 - protein, 12 aa, Artificial Sequence
  Synthetic
RPREDRVPHSCN

SEQ ID NO: 20 - protein, 11 aa, Artificial Sequence
  Synthetic
PREDRVPHSCN

SEQ ID NO: 21 - protein, 10 aa, Artificial Sequence
  Synthetic
REDRVPHSCN

SEQ ID NO: 22 - protein, 9 aa, Artificial Sequence
  Synthetic
EDRVPHSCN

SEQ ID NO: 23 - protein, 8 aa, Artificial Sequence
  Synthetic
DRVPHSCN

SEQ ID NO: 24 - protein, 7 aa, Artificial Sequence
  Synthetic
RVPHSCN

SEQ ID NO: 25 - protein, 6 aa, Artificial Sequence
  Synthetic
VPHSCN

SEQ ID NO: 26 - protein, 5 aa, Artificial Sequence
  Synthetic
PPSCN

SEQ ID NO: 27 - protein, 28 aa, Artificial Sequence
  Synthetic
PEHFSGRPREDRVPHSCNSITLTNLTPG

SEQ ID NO: 28 - protein, 5 aa, Artificial Sequence
  Synthetic
HHSCN

SEQ ID NO: 29 - protein, 5 aa, Artificial Sequence
  Synthetic
HPSCN

SEQ ID NO: 30 - protein, 5 aa, Artificial Sequence
  Synthetic
PHTCN

SEQ ID NO: 31 - protein, 5 aa, Artificial Sequence
  Synthetic
HHTCN

SEQ ID NO: 32 - protein, 5 aa, Artificial Sequence
  Synthetic
HPTCN

SEQ ID NO: 33 - protein, 5 aa, Artificial Sequence
  Synthetic
PHSNN

SEQ ID NO: 34 - protein, 5 aa, Artificial Sequence
  Synthetic
HHSNN

SEQ ID NO: 35 - protein, 5 aa, Artificial Sequence
  Synthetic
HPSNN

SEQ ID NO: 36 - protein, 5 aa, Artificial Sequence
  Synthetic
PHTNN

SEQ ID NO: 37 - protein, 5 aa, Artificial Sequence
  Synthetic
HHTNN

SEQ ID NO: 38 - protein, 5 aa, Artificial Sequence
  Synthetic
HPTNN

SEQ ID NO: 39 - protein, 5 aa, Artificial Sequence
  Synthetic
PHSKN

SEQ ID NO: 40 - protein, 5 aa, Artificial Sequence
  Synthetic
HHSKN

SEQ ID NO: 41 - protein, 5 aa, Artificial Sequence
  Synthetic
HPSKN

SEQ ID NO: 42 - protein, 5 aa, Artificial Sequence
  Synthetic
PHTKN

SEQ ID NO: 43 - protein, 5 aa, Artificial Sequence
  Synthetic
HHTKN

SEQ ID NO: 44 - protein, 5 aa, Artificial Sequence
  Synthetic
HPTKN

SEQ ID NO: 45 - protein, 5 aa, Artificial Sequence
  Synthetic
PHSCR

SEQ ID NO: 46 - protein, 5 aa, Artificial Sequence
  Synthetic
HHSCR

SEQ ID NO: 47 - protein, 5 aa, Artificial Sequence
  Synthetic
HPSCR

SEQ ID NO: 48 - protein, 5 aa, Artificial Sequence
  Synthetic
PHTCR

SEQ ID NO: 49 - protein, 5 aa, Artificial Sequence
  Synthetic
HHTCR

SEQ ID NO: 50 - protein, 5 aa, Artificial Sequence
  Synthetic
HPTCR

SEQ ID NO: 51 - protein, 5 aa, Artificial Sequence
  Synthetic
PHSNR

SEQ ID NO: 52 - protein, 5 aa, Artificial Sequence
  Synthetic
HHSNR

SEQ ID NO: 53 - protein, 5 aa, Artificial Sequence
  Synthetic
HPSNR

SEQ ID NO: 54 - protein, 5 aa, Artificial Sequence
  Synthetic
PHTNR

SEQ ID NO: 55 - protein, 5 aa, Artificial Sequence
  Synthetic
HHTNR

SEQ ID NO: 56 - protein, 5 aa, Artificial Sequence
  Synthetic
HPTNR

SEQ ID NO: 57 - protein, 5 aa, Artificial Sequence
  Synthetic
PHSKR

SEQ ID NO: 58 - protein, 5 aa, Artificial Sequence
  Synthetic
HHSKR

SEQ ID NO: 59 - protein, 5 aa, Artificial Sequence
  Synthetic
HPSKR

SEQ ID NO: 60 - protein, 5 aa, Artificial Sequence
  Synthetic
PHTKR

SEQ ID NO: 61 - protein, 5 aa, Artificial Sequence
  Synthetic
HHTKR

SEQ ID NO: 62 - protein, 5 aa, Artificial Sequence
  Synthetic
HPTKR

SEQ ID NO: 63 - protein, 5 aa, Artificial Sequence
  Synthetic
PHSCK

SEQ ID NO: 64 - protein, 5 aa, Artificial Sequence
  Synthetic
HHSCK

SEQ ID NO: 65 - protein, 5 aa, Artificial Sequence
  Synthetic
HPSCK

SEQ ID NO: 66 - protein, 5 aa, Artificial Sequence
  Synthetic
PHTCK

SEQ ID NO: 67 - protein, 5 aa, Artificial Sequence
  Synthetic
HHTCK

SEQ ID NO: 68 - protein, 5 aa, Artificial Sequence
  Synthetic
HPTCK

SEQ ID NO: 69 - protein, 5 aa, Artificial Sequence
  Synthetic
PHSNK

SEQ ID NO: 70 - protein, 5 aa, Artificial Sequence
  Synthetic
HHSNK

SEQ ID NO: 71 - protein, 5 aa, Artificial Sequence
  Synthetic
HPSNK

SEQ ID NO: 72 - protein, 5 aa, Artificial Sequence
  Synthetic
PHTNK

SEQ ID NO: 73 - protein, 5 aa, Artificial Sequence
  Synthetic
HHTNK

SEQ ID NO: 74 - protein, 5 aa, Artificial Sequence
  Synthetic
HPTNK

SEQ ID NO: 75 - protein, 5 aa, Artificial Sequence
  Synthetic
PHSKK

SEQ ID NO: 76 - protein, 5 aa, Artificial Sequence
  Synthetic
HHSKK

SEQ ID NO: 77 - protein, 5 aa, Artificial Sequence
  Synthetic
HPSKK

SEQ ID NO: 78 - protein, 5 aa, Artificial Sequence
  Synthetic
PHTKK

SEQ ID NO: 79 - protein, 5 aa, Artificial Sequence
  Synthetic
HHTKK

SEQ ID NO: 80 - protein, 5 aa, Artificial Sequence
  Synthetic
HPTKK

SEQ ID NO: 81 - protein, 5 aa, Artificial Sequence
  Synthetic
XXXXX

SEQ ID NO: 82 - protein, 5 aa, Artificial Sequence
  Synthetic
CHSRN

SEQ ID NO: 83 - protein, 5 aa, Artificial Sequence
  Synthetic
PCSRN

SEQ ID NO: 84 - protein, 5 aa, Artificial Sequence
  Synthetic
PHCRN

SEQ ID NO: 85 - protein, 5 aa, Artificial Sequence
  Synthetic
PHSRC

SEQ ID NO: 86 - protein, 5 aa, Artificial Sequence
  Synthetic
XHSXN

SEQ ID NO: 87 - protein, 5 aa, Artificial Sequence
  Synthetic
PHSXN

SEQ ID NO: 88 - protein, 4 aa, Artificial Sequence
  Synthetic
PSCN

SEQ ID NO: 89 - protein, 4 aa, Artificial Sequence
  Synthetic
HSCN

SEQ ID NO: 90 - protein, 4 aa, Artificial Sequence
  Synthetic
HTCN

SEQ ID NO: 91 - protein, 4 aa, Artificial Sequence
  Synthetic
PTCN

SEQ ID NO: 92 - protein, 4 aa, Artificial Sequence
  Synthetic
HSCR

SEQ ID NO: 93 - protein, 4 aa, Artificial Sequence
  Synthetic
PSCR

SEQ ID NO: 94 - protein, 4 aa, Artificial Sequence
  Synthetic
HTCR

SEQ ID NO: 95 - protein, 4 aa, Artificial Sequence
  Synthetic
PTCR

SEQ ID NO: 96 - protein, 4 aa, Artificial Sequence
  Synthetic
HSCK

SEQ ID NO: 97 - protein, 4 aa, Artificial Sequence
  Synthetic
PSCK

SEQ ID NO: 98 - protein, 4 aa, Artificial Sequence
  Synthetic
HTCK

SEQ ID NO: 99 - protein, 4 aa, Artificial Sequence
  Synthetic
PTCK